(19) United States
(12) Patent Application Publication
(10) Pub. No.: US 2021/0002384 A1
(43) Pub. Date: Jan. 7, 2021

(54) PROCESS FOR PRODUCING HU14.18K322A MONOCLONAL ANTIBODY

(71) Applicant: ST. JUDE CHILDREN'S RESEARCH HOSPITAL, Memphis, TN (US)

(72) Inventors: Michael M. Meagher, Cordova, TN (US); Muralidhar Reddivari, Collierville, TN (US)

(21) Appl. No.: 16/969,733

(22) PCT Filed: Feb. 15, 2019
(86) PCT No.: PCT/US2019/018169

§ 371 Date: Aug. 13, 2020

Related U.S. Application Data
(60) Provisional application No. 62/630,971, filed on Feb. 15, 2018.

(51) Int. Cl. C07K 16/30; C12N 5/071

(52) U.S. Cl. CPC C07K 16/3084; C12N 5/0602; C12N 2500/34; C07K 2317/41; C07K 2317/732

(57) ABSTRACT

A fed-batch process for producing Hu14.18K322A monoclonal antibody by culturing a mammalian cell culture in a culture medium including plant protein hydrolysates and a stable glucose concentration is provided, wherein said method yields a population of Hu14.18K322A monoclonal antibodies with increased titer and percentage of afucosylation.

### INTRODUCTION

This application is a continuation-in-part of U.S. Application Ser. No. 62/630,971, filed Feb. 15, 2018, the content of which is incorporated herein by reference in its entirety.

### BACKGROUND

Product quality attributes are critical for the functionality and manufacturability of therapeutic antibodies. They can be significantly influenced by a number of production process parameters, such as cell culture media. The composition of growth and feed media can influence antibody glycosylation, including the concentration of ammonia, glutamine, glucose, and metal ions. Thus, it is critical during media development and optimization to monitor and consider a culture medium's impact on glycosylation. For therapeutic antibodies whose mechanism of action includes antibody-dependent, cell-mediated cytotoxicity (ADCC), it is particularly important to reduce N-glycan fucosylation, which is known to influence ADCC activity. See, e.g., Shinkawa, et al. (2003) J. Biol. Chem. 278(5):3466-73; Niwa et al. (2004) Cancer Res. 64:2127-2133; Jefferis, et al. (1998) Immunol Rev. 163:59-76; Shields, et al. (2002) J. Biol. Chem. 277:26733-26740. Therefore, there is significant interest in obtaining a high antibody titer with appropriate fucosylation to increase therapeutic antibody efficacy.

One approach to addressing fucosylation has focused on the use of optimized host cell lines that produce defucosylated antibodies. See, e.g., Kanda, et al. (2006) Biotechnol. Bioeng. 94:680-688; Yamane-Ohnuki, et al. (2004) Biotechnol. Bioeng. 87:614-622; WO 2017/079165 and US 2009/0214528. In addition, process parameters such as pH, osmolality, temperature, and amino acid, lipid and ion (e.g., manganese) supplementation can modulate glycosylation of antibodies. See, e.g., Konno, et al. (2012) Cytotechnology 64:249-26; Hossler, et al. (2009) Glycobiology 19:936-949; Trummer, et al. (2006) Biotechnol. Bioeng. 94:1033-44; Miller, et al. (1988) Biotechnol. Bioeng. 32:947-965; WO 2013/114165; WO 2015/140700; US 2016/0362714; WO 2007/070315; US 2015/0344579; and WO 2017/120347.

While there exists a necessity for a cell culture process to provide a specific fucosylation profile, more importantly, the conditions used for modulating the fucosylated glycan content for a particular recombinant protein should be selected so as not to affect or significantly alter the amount or level of any other glycan profile. In addition, a combination of conditions for obtaining a specific fucosylation profile should have no significant impact on titer and/or productivity of the process. Thus, this invention provides a cell culture process for increasing both the quantity of an antibody and afucosylation of the antibody.

### SUMMARY OF THE INVENTION

A fed-batch process for producing a substantially afucosylated Hu14.18K322A monoclonal antibody in mammalian cell culture is provided. This method involves the steps of culturing mammalian host cells, which harbor a nucleic acid encoding Hu14.18K322A monoclonal antibody and are selected for producing substantially afucosylated Hu14.18K322A monoclonal antibody, in a cell culture medium comprising plant protein hydrolysates and a stable glucose concentration of 0.5 g/L to 1.5 g/L, thereby producing Hu14.18K322A monoclonal antibody in mammalian cell culture. In one embodiment, the fed-batch process further includes the step of purifying the Hu14.18K322A monoclonal antibody by, e.g., contacting the mammalian cell culture comprising the Hu14.18K322A monoclonal antibody with a protein A resin and eluting the Hu14.18K322A monoclonal antibody. A population of Hu14.18K322A monoclonal antibodies, e.g., having an antibody titer of at least about 400 mg/L, is also provided, wherein said antibodies are afucosylated (e.g., at least 55% afucosylation) and exhibit enhanced ADCC activity. In certain embodiments, the population of Hu14.18K322A monoclonal antibodies is provided in a pharmaceutical composition, wherein said antibodies are in admixture with a physiologically acceptable diluent, carrier, or excipient. The invention also provides a mammalian host cell harboring a nucleic acid encoding Hu14.18K322A monoclonal antibody and selected for producing substantially afucosylated Hu14.18K322A monoclonal antibody (e.g., at least 55% afucosylation) and a mammalian host cell deposited under American Type Culture Collection accession number ______ on Feb. 13, 2019.

### BRIEF DESCRIPTION OF THE DRAWINGS

FIG. 1 shows the glycan distribution of Hu14.18K322A monoclonal antibody produced by the method of this invention compared to a conventional preparation of Hu14.18K322A monoclonal antibody. Sample 1 and Sample 2 represent two individual clarified harvests of the Hu14.18K322A antibody.

FIG. 2 shows a plot of ADCC activity versus % afucosylation (% AF) of monoclonal antibody Hu14.18K322A compared to ch14.18 (a chimeric anti-GD2 antibody).

### DETAILED DESCRIPTION OF THE INVENTION

Hu14.18K322A is an antibody that contains fully human amino acid sequences for immunoglobulin G1 heavy and kappa light chains, and the complementarity-determining regions correspond to the antigen binding sequences of the murine 14.18 antibody. The resulting Hu14.18 antibody is approximately 98% derived from human genes, thereby making it less immunogenic. In addition, Hu14.18K322A has a single point mutation (K322A) designed to prevent activation of the complement cascade (see U.S. Pat. Nos. 7,432,357, 8,835,606 and 9,617,349, incorporated herein by reference in their entireties). In vitro analyses have shown that Hu14.18K322A retains the binding specificity and ADCC capabilities of ch14.18, with virtually no complement-dependent lysis. Furthermore, in vivo analyses in rats documented less dysesthesia with Hu14.18K322A than with ch14.18. Thus, Hu14.18K322A has the potential to cause less complement-mediated pain and fewer hypersensitivity reactions than ch14.18.

A process for the production of Hu14.18K322A antibody resulting in high antibody concentration with comparable or better in vivo activity has now been developed. The developed fed-batch process includes culturing the production clone in plant protein hydrolysates and controlling the concentration of glucose, the combination of which provides an increase in both the quantity of product (i.e., titer and highest ADDC activity) and quality of product (i.e., lowest percentage of afucosylated antibody, e.g., at least 55% afucosylation).

Accordingly, this invention provides methods and compositions for improving Hu14.18K322A antibody expression in cell culture, particularly mammalian cell culture. In particular, the invention includes improved fed-batch methods and compositions for promoting Hu14.18K322A monoclonal antibody production by adding culture media supplements, e.g., plant protein hydrolysates, to a basal culture medium. Specifically, this invention provides a method for producing Hu14.18K322A monoclonal antibody in a fed-batch process by culturing mammalian host cells harboring recombinant nucleic acids encoding Hu14.18K322A in a cell culture or basal medium containing plant protein hydrolysates and a stable glucose concentration of 0.5 to 1.5 g/L during Hu14.18K322A expression thereby producing Hu14.18K322A monoclonal antibody in mammalian cell culture. This invention also includes the purification and formulation of this antibody.

As described herein, “Hu14.18K322A” refers the 14.18 antibody having a single point mutation (K322A) designed to prevent activation of the complement cascade (see U.S. Pat. Nos. 7,432,357, 8,835,606 and 9,617,349). The Hu14.18K322A antibody of this invention can be a glycosylated or non-glycosylated immunoglobulin of any isotype or subclass and includes an antigen-binding region thereof that competes with the intact antibody for specific binding. The Hu14.18K322A antibody is preferably human or humanized and includes chimeric, multi-specific, and monoclonal antibodies or antigen binding fragments thereof. For the purpose of this invention, the term “antibody” is inclusive of, but not limited to, those that are prepared, expressed, created or isolated by recombinant means, such as antibodies isolated from a host cell transfected with nucleic acids that encode the Hu14.18K322A antibody. In particular embodiments, the Hu14.18K322A antibody is a humanized monoclonal antibody.

While various host cells can be used to produce the Hu14.18K322A antibody, mammalian host cells are preferred, and animal cells derived from primates such as human, monkey and the like, or animal cells derived from rodents such as mouse, rat, hamster and the like are more preferred. The cells belonging to mammals are preferably myeloma cells, ovarian cells, renal cells, blood cells, uterine cells, connective tissue cells, mammary cells, embryonic retinoblastoma cells, or cells derived therefrom, and more preferably cells selected from myeloma cells, myeloma cell-derived cells, ovarian cells, and ovarian cell-derived cells.

Examples of mammalian host cells include human cell lines such as HL-60 (ATCC No. CCL-240), HT-1080 (ATCC No. CCL-121), HeLa (ATCC No. CCL-2), 293 (ECACC No. 85120602), Namalwa (ATCC CRL-1432), Namalwa KJM-1 (Hosoi, et al. (1988) Cytotechnology 1:151), NM-F9 (DSM ACC2605, WO 2005/017130) and PER.C6 (ECACC No. 96022940, U.S. Pat. No. 6,855,544); monkey cell lines such as VERO (ATCC No. CCL-1651) and COS-(ATCC No. CRL-1651); mouse cell lines such as C1271 (ATCC No. CRL-1616), Sp2/0-Ag14 (ATCC No. CRL-1581), and NIH3T3 (ATCC No. CRL-1658), NSO (ATCC No. CRL-1827); rat cell lines such as Y3 Ag 1.2.3. (ATCC No. CRL-1631), YO (ECACC No. 85110501) and YB2/0 (ATCC No. CRL-1662); hamster cell lines such as CHO-K1 (ATCC No. CCL-61), CHO/dhfr-(ATCC No. CRL-9096), CHO/DG44 (Urlaub & Chasin (1980) Proc. Natl. Acad. Sci. USA 77:4216) and BHK21 (ATCC No. CRL-10); dog cell lines such as MDCK (ATCC No. CCL-34), and the like.

Examples of the myeloma cell or myeloma cell-derived cells may include Sp2/0-Ag14, NSO, Y3 Ag 1.2.3., Y0 or YB2/0 and the like. Examples of the ovarian cells or ovarian cell-derived cells may include CHI-K1, CHO/dhfr-, CHO/DG44 and the like. Further, examples of the renal cells may include 293, VERO, COS-7, BHK21, MDCK and the like. Examples of the blood cells may include HL-60, Namalwa, Namalwa KJM-1, NM-F9 and the like. Examples of the uterine cells may include HeLa and the like. Examples of the connective tissue cells may include HT-1080, NIH3T3 and the like. Examples of the mammary cells may include C12711 and the like. Examples of the embryonic retinoblastoma cells may include PER.C6 and the like.

Mammalian host cells having the ability to produce the antibody of the present invention may include fusion cells prepared to produce the antibody or the like. Further, mammalian cells that are mutated to produce the antibody, mammalian cells that are mutated to have an increased expression level of the antibody or the like are also included in the mammalian cells of the present invention.

The mammalian host cells producing the antibody of the present invention preferably include recombinant mammalian host cells that are transformed with recombinant nucleic acids (e.g., a vector) encoding the Hu14.18K322A antibody. Recombinant nucleic acids encoding the Hu14.18K322A antibody are known in the art and include, e.g., the expression plasmid pdHL7s-hu14.18 disclosed in U.S. Pat. No. 7,432,357, incorporated herein by reference. The transformed cells for expressing the antibody may be obtained by introduction of the recombinant vector into the mammalian host cells by conventional methods. See, e.g., Kaufman (1990) Large Scale Mammalian Cell Culture, pp. 15-69. Additional protocols using commercially available reagents, such as the cationic lipid reagents LIPOFECTAMINE™ LIPOFECTAMINE™-2000, or LIPOFECTAMINE™-plus can be used to transfect cells (Feigner, et al. (1987) Proc. Natl. Acad. Sci. USA 84:7413-7417). In addition, electroporation or bombardment with microprojectiles coated with nucleic acids can be used to transfect mammalian cells using procedures, such as those in Sambrook et al. (1989) Molecular Cloning: A Laboratory Manual, 2nd ed. Vol. 1-3, Cold Spring Harbor Laboratory Press. Selection of stable transformants can be performed using methods known in the art, such as, for example, resistance to compounds such as G418 and hygromycin B.

A mammalian host cell harboring a nucleic acid encoding Hu14.18K322A monoclonal antibody and selected for producing substantially afucosylated Hu14.18K322A monoclonal antibody is also provided by this invention. In certain embodiments, the antibody produced by the mammalian host cell is at least 55% afucosylated. An exemplary mammalian host cell line (clone 134) is deposited with the American Type Culture Collection, 10801 University Blvd., Manassas, Va. 20110 under ATCC accession number ______ on Feb. 13, 2019.

As indicated, the instant method is carried out using a fed-batch process. “Fed-batch process,” as used herein, is a process where the cultivation is started by inoculating cells in a cell culture medium or basal medium and where additions of various additives are performed during the cultivation. Fed-batch culture is a widely-practiced culture method for large scale production of proteins from mammalian cells. See, e.g., Chu & Robinson (2001) Current Opin. Biotechnol. 12:180-87. A fed-batch culture of mammalian cells is one in which the culture is fed, either continuously or periodically, with a concentrated basal medium and various additives. Feeding can occur on a predetermined schedule of, for example, every day, once every two days, once every three days, etc. When compared to a batch culture, in which no feeding occurs, a fed-batch culture can produce greater amounts of protein. See, e.g., U.S. Pat. No. 5,672,502. In certain embodiments, the fed-batch culture of the present method produces an antibody titer of at least 1.5 g/L. In another embodiment, a titer of at least 2 g/L is produced. In another embodiment, at least 4 g/L of the antibody is produced. In still another embodiment, at least 5 g/L of the antibody is produced. In a further embodiment, the invention provides a method for producing about 6 g/L of an antibody.

The method according to the present invention may be carried out in a single-phase process or multiple phase process. Compared to a single-phase process, the multiple phase process refers to culturing of the cells in two or more distinct phases. For example, cells may be cultured first in one or more growth phases, under environmental conditions that maximize cell proliferation and viability, then transferred to a production phase, under conditions that maximize protein production. “Growth phase,” as used herein, refers to the period during which cultured cells are rapidly dividing and increasing in number. During growth phase, cells may be generally cultured in a medium and under conditions designed to maximize cell proliferation. “Production phase” refers to a period during which cells are producing maximal amounts of a recombinant protein. A production phase is characterized by less cell division than during a growth phase, and may also include the use of medium and culture conditions designed to maximize polypeptide production.

In a commercial process for production of a protein by mammalian cells, there are commonly multiple, for example, at least about 2, 3, 4, 5, 6, 7, 8, 9, or 10 growth phases that occur in different culture vessels preceding a final production phase. The growth and production phases may be preceded by, or separated by, one or more transition phases. In multiple phase processes, the method according to the present invention can be employed at least during the production phase, although it may also be employed in a preceding growth phase. A production phase can be conducted at large scale. Typically, cell culture is performed under sterile, controlled temperature and atmospheric conditions in bioreactors. A bioreactor is a device used to culture cells in which environmental conditions such as temperature, atmosphere, agitation, and/or pH can be monitored, adjusted and controlled. A large-scale process can be conducted in a volume of at least about 100, 500, 1000, 2000, 3000, 5000, 7000, 8000, 10,000, 15,000, 20,000 liters.

In some embodiments, the growth phase is carried out at a temperature in the range of about 33° C. to about 38° C. (preferably about 37° C.). Similarly, the production phase is carried out at a temperature in the range of about 28° C. to about 38° C., preferably in the range of about 33° C. to about 37° C. In particular embodiments, the temperature of the culture is 37° C.

The step of culturing may optionally include chemical inducers of protein production, such as, for example, caffeine, butyrate, and hexamethylene bisacetamide (HMBA). If inducers are added, they can be added from one hour to five days after the start of the production phase.

In accordance with the present fed-batch process, the mammalian cells are minimally provided a combination feed solution including a basal medium, glucose, and a combination of plant protein hydrolysates. For the purposes of this invention, a “basal medium” or “cell culture medium” is a medium suitable for growth of animal cells, such as mammalian cells, in in vitro cell culture. Basal media formulations are well known in the art. Typically, basal media supplies standard inorganic salts, such as zinc, iron, magnesium, calcium and potassium, as well as trace elements, vitamins, an energy source, a buffer system, and essential amino acids. The basal medium may or may not contain serum, peptone, and/or proteins. Various basal media, including serum-free and defined culture media, are commercially available. For example, any one or a combination of the following cell culture media can be used: RPMI-1640 Medium, RPMI-1641 Medium, Dulbecco's Modified Eagle's Medium (DMEM), Minimal Essential Medium (MEM), Basal Medium Eagle (BME), Minimum Essential Medium Eagle, F-12K Medium, Ham's F12 or F-10 Medium, DME/F12, α-Minimal Essential Medium (α-MEM), Glasgow's Minimal Essential Medium (G-MEM), PF CHO (SAFC Biosciences), POWERCHO™ 2 (Lonza), ZAP-CHO (Invitria), CD CHO, CD OptiCHO™ and CHO-S-SFMII (Invitrogen), ProCHO™ (Lonza), CDM4CHO (Hyclone), Iscove's Modified Dulbecco's Medium, McCoy's 5A Medium, Leibovitz's L-15 Medium, and serum-free media such as Hybridoma Serum Free Medium (HSFM) and EX-CELL™ 300 Series (JRH Biosciences, Lenexa, Kans.), among others.

A basal medium may be supplemented with additional or increased concentrations of ingredients depending on the requirements of the cells to be cultured and/or the desired cell culture parameters. The term “ingredient” refers to any compound, whether of chemical or biological origin, that can be used in cell culture media to maintain or promote the growth of proliferation of cells. The terms “component,” “nutrient” and ingredient” are used interchangeably and are all meant to refer to such compounds. Typical ingredients that are used in cell culture media include amino acids, salts, metals, sugars, lipids, nucleic acids, hormones, vitamins, fatty acids, proteins and the like. Other ingredients that promote or maintain cultivation of cells ex vivo can be selected by those of skill in the art within the scope of the invention, and in accordance with the particular need.

In a preferred embodiment, the cell culture media of the invention is serum-free, protein-free, and/or peptone-free. Cell culture medium is considered “serum-free” when said medium contains no serum (e.g., fetal bovine serum (FBS), horse serum, goat serum, or any other animal-derived serum known to one skilled in the art). “Protein-free” applies to cell culture media free from exogenously added protein, such as transferrin, protein growth factors IGF-1, or insulin. Protein-free media may or may not contain peptones. “Peptone-free” applies to basal media which contains no exogenous protein hydrolysates such as animal and/or plant protein hydrolysates.

In some embodiments, the basal medium is modified to remove certain non-nutritional components found in a standard or conventional basal medium, such as various inorganic and organic buffers, surfactant(s), and sodium chloride. Removing such components from basal cell medium allows an increased concentration of the remaining nutritional components, and may improve overall cell growth and protein expression. In some embodiments, a modified basal medium excludes any, if not all, of the following ingredients: sodium bicarbonate, a buffer, mono-basic sodium phosphate, di-basic sodium phosphate, and a surfactant. See, U.S. Pat. No. 9,234,032. These ingredients are commonly found in commercial basal cell media and may be removed by commercial media services such as SAFC (formerly JRH Bioscience), Invitrogen, Atlanta Biologicals, and Lonza. Alternatively, one of ordinary skill in the art can prepare a modified basal cell medium according to standard methods for making basal cell media, wherein one or more of sodium bicarbonate, a buffer, mono-basic sodium phosphate, di-basic sodium phosphate, and a surfactant are omitted.

When using a modified basal medium, it is preferable that the one or more components omitted from the basal medium are added back to the cell culture medium during growth and/or production phases. When added to the modified basal medium, preferably the following amounts of components are used: about 1 to 3 g/kg sodium bicarbonate; about 1 to 3 g/kg buffer (e.g., N-[2-hydroxyethyl]piperazine-N′-[2-ethansul-phonic acid] (HEPES)); about 0.01 to 0.1 g/kg NaH₂PO₄—H₂O; about 0.1 to 0.1 g/kg Na₂HPO₄-7H₂O; and about 0.1 to 2 g/kg surfactant (e.g., block copolymers based on ethylene oxide and propylene oxide sold under the trademark PLURONIC® F-68).

Notably, the buffer is included to help maintain the cell culture medium at a desired pH. In one embodiment, the pH of the cell culture medium ranges from 6.0 to 8.0; 6.5 to 7.5; or 6.8 to 7.3. Numbers intermediate to these pH values, e.g., 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, and 8.0, as well as all other numbers recited herein, are also intended to be part of this invention. As applied to any of the ranges disclosed herein, said ranges of values using a combination of any of the above recited values as upper and/or lower limits are intended to be included in the scope of the invention.

In certain embodiments, the basal medium omits an osmolarity regulator. The term “osmolality,” as used herein, is defined as a measure of the osmoles of solute per kilogram of solvent (mOsm/kg) and may include ionized or non-ionized molecules. The osmolality may change during the cell culture process by, e.g., addition of feed, salts, additives or metabolites. Preferably, the cell culture medium at the time of antibody production (i.e., during production phase) has an osmolality ranging from about 300 to 500 mOsm/kg or more preferably about 400 to 430 mOsm/kg, as well as numbers intermediate thereto. In some embodiments, the osmolarity regulator is NaCl, KCl, or KNO₃. In certain embodiments, the osmolarity regulator is NaCl. In one embodiment, osmolarity regulator is added to the cell culture medium at a final concentration of between 0 g/L to 10 g/L. In another embodiment, the final concentration of the osmolarity regulator is 0 g/L to 6.5 g/L. Ranges of values using a combination of any of the above recited values as upper and/or lower limits are intended to be included in the scope of the invention.

In accordance with this invention, the fed-batch process includes the addition of at least one plant protein hydrolysate to the basal medium. The term “hydrolysate” includes any enzymatic digest, particularly a specialized type of extract prepared by treating plant components with at least one enzyme capable of breaking down the components of the plant into simpler forms (e.g., into a preparation comprising mono- or disaccharides and/or mono-, di- or tripeptides). An “hydrolysate” can be further enzymatically digested, for example by papain, and/or formed by autolysis, thermolysis and/or plasmolysis. In certain embodiments, the plant protein hydrolysate is an enzymatically hydrolyzed protein hydrolysate of soy, wheat, cotton, whey, pea, chickpea or cotton. Hydrolysates used in the media of the invention are commercially available, including, for example, protein hydrolysates sold under the trademark HYPEP® 1510 or HY-SOY® from sources such as Quest International (Norwich, N.Y.), Soy Hydrolysate UF from SAFC Biosciences, HYQ® Soy Hydrolysate from HyClone Media or soy peptones (enzymatic digests of soybean meal/flour) such as soytone, phytone, phytone peptone, or a combination thereof. Preferably, a plant protein hydrolysate is included in the cell culture medium in an amount of about 6 to 12 g/L, e.g., about 8 to 10 g/L. In one embodiment, the cell culture medium includes about 6 to 12 g/L soy protein hydrolysate, e.g., about 8 to 10 g/L soy protein hydrolysate. In some embodiments, the plant protein hydrolysate is added to the cell culture medium in an amount that does not exceed 25 g/L in the feed. In particular embodiments, initial growth medium includes 2% soytone and 2% phytone. In further embodiments, the culture medium added during the production phase includes 8-10% soytone and 8-10% phytone.

In accordance with this invention, the fed-batch process may also include the addition of cysteine or a cysteine derivative, such as N-acetyl cysteine, to the basal medium. In particular embodiments, cysteine is included in the cell culture medium in an amount of about 0.1 to 10 mM, or more preferably about 1 to 7 mM cysteine.

In some embodiments, the plant protein hydrolysates and optional cysteine are provided as independent feeds. In other embodiments, the plant protein hydrolysate and cysteine are provided together in a hydrolysate enrichment solution, which is added as an independent feed. The independent plant protein hydrolysate and cysteine feeds or hydrolysate enrichment solution can begin just prior to or at the start of the antibody production phase. The independent feeds can be accomplished by fed-batch to the cell culture medium on the same or different days as the basal medium. The independent feeds can be added to the cell culture medium after one or more days, and can also be added repeatedly during the course of the production phase. For example, the production phase can last from 7 days to as long as 8, 9, 10, 11, 12, 13, or 14 days or longer and be supplemented with the independent feeds immediately and/or on days 2, 3, 4, 5, 6, 7, 8, 9, 10, 11, 12, 13 and/or later days of the production phase.

An energy source is also added to the cell culture medium of the invention. Preferably, the energy source is a monosaccharide. Examples of monosaccharides which may be used in the cell culture medium include glucose (e.g., D-glucose), maltose, mannose, galactose and fructose. In one embodiment, glucose is added to the cell culture medium at a final concentration ranging from 0.5-4.0 g/L. In another embodiment, glucose is added to the cell culture medium at a final concentration of no greater than 4.0 g/L. In certain embodiments, glucose is added to the cell culture medium at a final concentration of about 0.5 to 1.5 g/L and most preferably 1.0 g/L. Numbers intermediate to the recited glucose concentrations, e.g., 0.5, 0.75, 1.0, 1.25, 1.5, 2.0, 2.5, 3.0, 3.5, 3.6, 3.7, 3.8, 3.9, and 4.0, as well as numbers intermediate thereto, are also intended to be part of this invention. Ranges of values using a combination of any of the above recited values as upper and/or lower limits are also intended to be included in the scope of the invention. Preferably the glucose concentration is monitored in real-time and maintained during the production phase at a stable concentration in the range of about 0.5 g/L to 1.5 g/L, or more preferably at about 1 g/L±0.1 g/L to facilitate antibody production and maintain cell viability. A “stable glucose concentration” means that the glucose concentration does not go below about 0.5 g/L or above about 1.5 g/L. Notably, glucose concentrations of 4 to 5 g/L were found to adversely affect viability and productivity of Hu14.18K322A antibody. Ideally, a feed-forward algorithm is used, which is based on known glucose consumption rates during the different phases of the culture, so that the desired glucose concentration is maintained. Preferably, the feed-forward approach accounts for 100% of the glucose consumption and a back-up closed-loop glucose control system, with a glucose lower limit setpoint of 0.5 g/L that would trigger the glucose control system and maintain at 1 g/L. The glucose control system can be turned on or off as needed.

The cell culture medium of the invention may further include glutamine, e.g., L-glutamine. Suitable sources of L-glutamine are available from various commercial sources, such as GIBCO. In some embodiments, the glutamine is provided in the cell culture medium in an amount of about 0.1 to 0.5 g/kg.

The cell culture medium of the invention may further include glutathione. In one embodiment, 0.4 mg/L to 1.65 mg/L glutathione is added to the cell culture medium.

In another embodiment, the cell culture medium includes a recombinant growth factor such as insulin or a recombinant analog, IGF-1, or a combination of insulin and IGF-1. In one embodiment, 4 mg/L to 13 mg/L insulin or a recombinant analog is added. In another embodiment, 25 ng/L to 150 ng/L IGF-1 is added. In yet another embodiment, 50 ng/L to 100 ng/L IGF-1 is added. In still another embodiment, 25 ng/L to 150 ng/L IGF-1 is supplemented to the insulin. In one embodiment, 50 ng/L to 100 ng/L IGF-1 is supplemented to the insulin.

In still another embodiment, the cell culture medium includes an inorganic iron source, e.g., ferric citrate. In one embodiment, 10 mL/L or 122 mg/L ferric citrate is added. In yet another embodiment, the ferric citrate is held to a concentration of 122 mg/L.

The cell culture medium of the invention may also include non-ferrous metal ions. Examples of non-ferrous metal ions include, but are not limited to, chloride and sulfate salts, potassium, magnesium, cupric, selenium, zinc, nickel, manganese, tin, cadmium, molybdate, vanadate, and silicate.

The cell culture medium of the invention may also include vitamins and enzyme co-factors. Examples of such vitamins and enzyme co-factors include, but are not limited to, PABA (p-Aminobenzoic Acid), Vitamin K (Biotin), Vitamin B5 (D-Calcium Pantothenate), Folic Acid, I-Inositol, Niacinamide (Nicotinic Acid Amide), Vitamin B6 (Pyridoxine HCl), Vitamin B2 (Riboflavin), Vitamin B1 (Thiamine), and Vitamin B12 (Cyanocobalamin). Alternatively, vitamin C (L-Ascorbic Acid) may be added to the media. Choline Chloride may also be added, it is usually considered a vitamin but it may also be considered a lipid factor.

Additionally, the cell culture medium of the invention may also include lipid like factors. Examples of lipid factors include choline chloride and phosphatidylcholine. An aid in lipid production, e.g., an alcohol amine like ethanolamine, may also be included.

Optionally, the cell culture medium may include methotrexate. Examples of amounts of methotrexate used in the cell culture media include about 100 nM to 5000 nM methotrexate. Numbers intermediate to the recited methotrexate molarity, e.g., 100, 200, 300, 400, 500, 600, 700, 800, 900, 1000, 1200, 1400, 1600, 1800, 2000, 2200, 2400, 2600, 2800, 3000, 3200, 3400, 3600, 3800, 4000, 4200, 4400, 4600, 4800, and 5000 nM, as well as numbers intermediate thereto, are also intended to be part of this invention. Ranges of values using a combination of any of the above recited values as upper and/or lower limits are also intended to be included in the scope of the invention.

Using the method of this invention, it has been found that titer of Hu14.18K322A antibody is increased. In particular embodiments, the method of producing the Hu14.18K322A antibody provides a titer of the antibody that is at least 10% greater than a control mammalian cell culture. In some embodiments, the antibody titer of the mammalian cell culture is improved at least 25%, 40%, 50%, 75%, or 90% over the control mammalian cell culture. In other embodiments, the antibody titer of the mammalian cell culture is at least 90%, 150% or 250% greater than the control mammalian cell culture. In still other embodiments, the antibody titer is at least about 400 mg/L, about 600 mg/L, about 800 mg/L or higher.

Using the method of this invention, it has been found that the percentage of afucosylated Hu14.18K322A antibody is increased compared to conventional production methods. Notably, the afucosylated Hu14.18K322A antibody is produced in the presence or absence of a fucosylation inhibitor such as 2F-peracetyl-fucose or betaine. An “afucosylated Hu14.18K322A antibody” refers to a Hu14.18K322A antibody that lacks fucose in its constant region glycosylation. Glycosylation of human IgG1 or IgG3 occurs at Asn297 as core fucosylated biantennary complex oligosaccharide glycosylation terminated with up to 2 Gal residues. In some embodiments, an afucosylated antibody lacks fucose at Asn297. Exemplary afucosylated species include the GO glycan, Gla glycan, Gib glycan, G2 glycan, Man 3 glycan, Man 4 glycan, Man 5 glycan, Man 6 glycan, Man glycan, Man 8 glycan, and/or Man 9 glycan, or any combinations thereof. Preferably, the afucosylated species is the GO glycan. In particular embodiments, the present method of producing the Hu14.18K322A antibody provides an increased percentage of afucosylated Hu14.18K322A antibodies as compared to Hu14.18K322A antibodies produced in a control mammalian cell culture, wherein the increase in said afucosylated Hu14.18K322A antibodies is by at least about 5%, 10%, 15%, 20% or 25%. In other embodiments, a composition containing a plurality of Hu14.18K322A antibodies is considered to be “substantially afucosylated” if at least 55%, 60%, 65%, 70%, 75%, 80% or 85% of the total amount of antibody expressed by the cells is afucosylated. Methods of measuring fucose include any methods known in the art. See, e.g., Wuhrer, et al. (2005) J. Chromatog. B 825(2):124-133; Ruhaak (2010) Anal. Bioanal. Chem. 397:3457-3481; and Geoffrey, et al. (1996) Anal. Biochem. 240:210-226.

For the purposes of this invention, a “control mammalian cell culture” includes culturing mammalian cells harboring a nucleic acid encoding Hu14.18K322A monoclonal antibody in a conventional cell culture medium under culture conditions and osmolality in the absence of a plant protein hydrolysates and cysteine.

Ideally, a Hu14.18K322A antibody produced in the accordance with the present method has enhanced antibody-dependent cell-mediated cytotoxicity (ADCC) activity. An antibody having an “enhanced ADCC activity” refers to an antibody that is more effective at mediating ADCC in vitro or in vivo compared to the parent antibody, wherein the antibody and the parent antibody differ in at least one structural aspect, and when the amounts of such antibody and parent antibody used in the assay are essentially the same. In some embodiments, the antibody and the parent antibody have the same amino acid sequence, but the antibody is afucosylated while the parent antibody is fucosylated. In some embodiments, ADCC activity will be determined using the in vitro ADCC assay, but other assays or methods for determining ADCC activity, e.g., in an animal model etc., are contemplated.

After producing the Hu14.18K322A monoclonal antibody, the antibody is recovered or collected and purified or partially purified from the culture (e.g., from culture medium or cell extracts) using known processes. Fractionation procedures can include but are not limited to one or more steps of filtration, centrifugation, precipitation, phase separation, affinity purification, gel filtration, ion exchange chromatography, hydrophobic interaction chromatography (HIC; using such resins as phenyl ether, butyl ether, or propyl ether), HPLC, or some combination of above.

For example, the purification of the Hu14.18K322A monoclonal antibody can include a protein A or Protein G resin, which will bind to the polypeptide; and one or more steps involving elution. Polypeptides can be removed from an affinity column using conventional techniques, e.g., in a high salt elution buffer and then dialyzed into a lower salt buffer for use or by changing pH or other components depending on the affinity matrix utilized, or can be competitively removed using the naturally occurring substrate of the affinity moiety. The desired degree of final purity depends on the intended use. The methods and compositions of the invention are suitable for therapeutic uses. Thus, a relatively high degree of purity is desired when the antibody is to be administered in vivo. In such a case, the antibody is purified such that no polypeptide bands corresponding to other polypeptides are detectable upon analysis by SDS-polyacrylamide gel electrophoresis (SDS-PAGE). It will be recognized by one skilled in the pertinent field that multiple bands corresponding to the antibody can be visualized by SDS-PAGE, due to differential glycosylation, differential post-translational processing, and the like. Most preferably, the antibody of the invention is purified to substantial homogeneity, as indicated by a single polypeptide band upon analysis by SDS-PAGE. The polypeptide band can be visualized by silver staining, Coomassie blue staining, or (if the polypeptide is radiolabeled) by autoradiography.

The invention also optionally encompasses further formulating the Hu14.18K322A monoclonal antibody in a pharmaceutical composition. By the term “formulating” is meant that the antibody can be buffer exchanged, sterilized, bulk-packaged and/or packaged for a final user. Such pharmaceutical compositions can include an effective amount of the Hu14.18K322A monoclonal antibody, in combination with other components such as a physiologically acceptable diluent, carrier, or excipient. The term “physiologically acceptable” means a non-toxic material that does not interfere with the effectiveness of the biological activity of the active ingredient(s).

In light of the findings presented herein for the production of Hu14.18K322A monoclonal antibody, one of ordinary skill in the art will appreciate that the method of this invention will also be useful for producing other antibodies with similar characteristics to that of the Hu14.18K322A monoclonal antibody.

The following non-limiting examples are provided to further illustrate the present invention.

### Example 1: Expression of Hu14.18K322A Antibody

An expression plasmid that expresses the heavy and light chains of the Hu14.18K322A is described in U.S. Pat. No. 7,432,357. Briefly, the Hu14.18K322A expression plasmid pdHL7-hu14.18:pdHL7 was derived from pdHL2 (Gillies, et al. (1989) J. Immunol. Methods 125:191-202), and uses the cytomegalovirus enhancer-promoter for the transcription of both the immunoglobulin light and heavy chain genes.

Electroporation is used to introduce the DNA encoding the Hu14.18K322A antibody described above into Chinese hamster ovary (CHO) cells or rat hybridoma cells YB2/0. To perform electroporation, cells are grown in Dulbecco's modified Eagle's medium supplemented with 10% heat-inactivated fetal bovine serum, 2 mM glutamine and penicillin/streptomycin. About 5×10⁶ cells are washed once with PBS and resuspended in 0.5 ml PBS. Ten micrograms of linearized plasmid DNA encoding the modified Hu14.18K322A antibody are then incubated with the cells in a Gene Pulser Cuvette (0.4 cm electrode gap, BioRad, Hercules, Calif.) on ice for 10 minutes. Electroporation is performed using a Gene Pulser (BioRad, Hercules, Calif.) with settings at 0.25 V and 500 pF. Cells are allowed to recover for 10 minutes on ice, after which they are resuspended in growth medium and plated onto two 96 well plates.

Stably transfected clones are selected by their growth in the presence of methotrexate (MTX). Specifically, parent clone #108-334 was identified from subcloning by limiting dilutions. Subcloning was carried out by plating cells at 8, 4, 2, 1, 0.5 and 0.25 cells/well in 96-well plates containing DMEM supplemented with 10% FBS, 1 mM sodium pyruvate, 2 mM glutamine, penicillin/streptomycin, and 50 nM MTX. When subclones appeared on the plates containing 0.25 cell/well, 0.5 cell/well and 1 cell/well, the plates containing at least 2 cells/well were discarded. The subclones in the wells were inspected under the microscope to ensure that there was only one visible clone in the well. A total of 112 subclones from six 96-well plates containing 0.25 cell/well, six 96-well plates containing 0.5 cell/well and six 96-well plates containing 1 cell/well were picked and antibody expression levels in the supernatants were assayed by anti-human Fc ELISA. The best subclones were #108-4 and #108-334, producing approximately 89 μg/ml and 106 μg/ml, respectively, in 75-cm² T flasks (by rPA analysis). Clone #108-334 was adapted to serum-free media, i.e., HSFM and 52 nM MTX, by a progressive reduction of serum. A master cell bank was produced by passaging clone #108-334 in HSFM media with 52 nM MTX and formulating the cells at 1×10⁷ cells/mL in Recovery™ Cell Culture Medium with storage in the vapor phase of liquid nitrogen.

A YB2/0-based production clone #134 was obtained by treating parent clone #108-334 with a step-wise increase in MTX concentration to a final concentration of 1,000 nM over a 4-week period. Clones were maintained in culture media containing 1,000 nM MTX for another 4 to 5 weeks and fed media as needed. A sample of this population of cells, that survived 1,000 nM MTX, was used for the next round of clonal selection.

The next step was to screen 3,360 individual 1,000 nM MTX-resistant clones for their ability to survive single-cell selection in HSFM with 6 mM Glutamax™, 2 g/L soytone and 2 g/L phytone in 1000 nM MTX. It was this selection process that resulted in production clone #134.

A master cell bank of production clone #134 was produced by growing the cells in HSFM with 6 mM Glutamax™, 2 g/L soytone and 2 g/L phytone in 1000 nM MTX. Cells were thawed, centrifuged, resuspended in growth medium and seeded into a suspension flask at 2-3×10⁵ cells/mL. Cells were passaged once the cell density reached 1-2×10⁶ cells/mL and passaging continued until there was 1-2×10⁹ cells. The cells were centrifuged and resuspended at 1×10⁷ cells/mL in fresh HSFM with 6 mM Glutamax™, 2 g/L soytone and 2 g/L phytone in 1000 nM MTX and 5% DMSO. The cells were subsequently frozen using a CryoMed™ controlled-rate freezer and stored in vapor-phase of liquid nitrogen.

### Example 2: Production of Hu1418 K322A by Fed-Batch Process

Hu14.18K322A producing cells were maintained in HSFM supplemented with 6 mM Glutamax™, 2 g/L soytone and 2 g/L phytone hydrolysate and either 52 nM MTX for parent clone #108-334 or 1000 nM MTX for production clone #134. The first step in the production of Hu14.18K322A was a standard inoculum seed train to generate cells to inoculate the production reactor. A vial from the master cell bank was thawed and cells were inoculated at a viable cell density of 0.2-0.3×10⁶ cells/mL in suspension. After reaching a viable cell density of 1-1.5×10⁶ cells/mL, these cells were used to inoculate the subsequent bioreactor at a cell density of 0.2-0.3×10⁶ cells/mL. The bioreactor for generating cells to seed the production bioreactor ranged from a small 50 mL shaking flask in a CO₂ incubator to a fully controlled bioreactor. Once the production bioreactor was inoculated at 0.2-0.3×10⁶ cells/mL, the reactor was cultured for 72 hours before the fed-batch phase was initiated. All inoculum seed cultures were grown at 37° C. and the dissolved oxygen (DO) level was set to 50% of air saturation. The pH was maintained at 6.9±0.03.

Impact of Soytone and Phytone on Antibody Production. After 72 hours, the Feed was started via pulsed modulated feeding. The Feed was composed of only HSFM medium and 6 mM Glutamax™ (i.e., no soytone or phytone) and was provided at a constant rate of 3.5-4.5 mL of Feed/hour/L of the starting volume of the bioreactor. Glucose was allowed to decrease to 1 g/L via cellular metabolism and was maintained at this value by a separate glucose feed; both Feed and glucose addition were controlled by a bioreactor control system algorithm. After 210 hours of total elapse culture time (TECT), which started when the inoculum was transferred into the production bioreactor, hu14.18K322A titers of 130 mg/L were achieved with an afucosylation percentage (% AF) of 54% for clone #108-334. Reactors were clarified via depth filtration followed by sterile (0.2 mm) filtration and used for downstream processing.

The impact of g/L of soytone and g/L phytone (in the Feed and the clone) on hu14.18K322A yield, % AF and TECT is summarized in Table 1.

TABLE 1

| | Soytone | Phytone | hu14.18K322A | | TECT |
|---|---|---|---|---|---|
| Clone | (g/L) | (g/L) | (mg/L) | % AF | (h) |
| 108-334 | 0 | 0 | 130 | 54 | 210 |
| 108-334 | 2 | 2 | 236 | 42 | 312 |
| 108-334 | 4 | 4 | 314 | 24 | 304 |
| 108-334 | 6 | 6 | 340 | 21 | 304 |
| 108-334 | 8 | 8 | 362 | 22 | 304 |
| 134 | 6 | 6 | 497 | 76 | 304 |
| 134 | 8 | 8 | 507 | 76 | 304 |
| 134 | 10 | 10 | 774 | 82 | 326 |
| 134 | 15 | 15 | 693 | 80 | 279 |
| 134 | 20 | 20 | 512 | 82 | 255 |

The bioreactor runs were terminated when the cell viability dropped below 1×10⁶ cells/mL. These results show that increasing the amount of soytone and phytone in the Feed had a direct impact on hu14.18K322A yield and quality. Amino acid analysis of cell culture supernatant showed that lower soytone/phytone concentrations, i.e., 6 g/L, were low in several essential amino acids, such as cysteine, while soytone/phytone levels above 8 g/L, i.e., 10 g/L and 15 g/L, had adequate levels of essential amino acids at the conclusion of the run. The results showed that there was an optimum level of soytone/phytone for hu14.18K322A production.

Impact of Temperature Shift on Antibody Production. The temperature of the bioreactor was reduced from 37° C. to 33° C. over 24 hours on days 4, 5, and 6 of TECT. The Feed medium was set at 10 g/L of soytone and phytone and the antibody production was achieved using clone #134. The impact of temperature shifts on antibody production is summarized in Table 2.

TABLE 2

| | hu14.18K322A | | TECT |
|---|---|---|---|
| Day of Temperature Shift | (mg/L) | % AF | (h) |
| No Temperature Shift | 871 | 82.0 | 308 |
| Day 4 | 698 | 80.6 | 308 |
| Day 5 | 820 | 80.6 | 308 |
| Day 6 | 856 | 78.8 | 308 |

### Example 3: Glycan Distribution

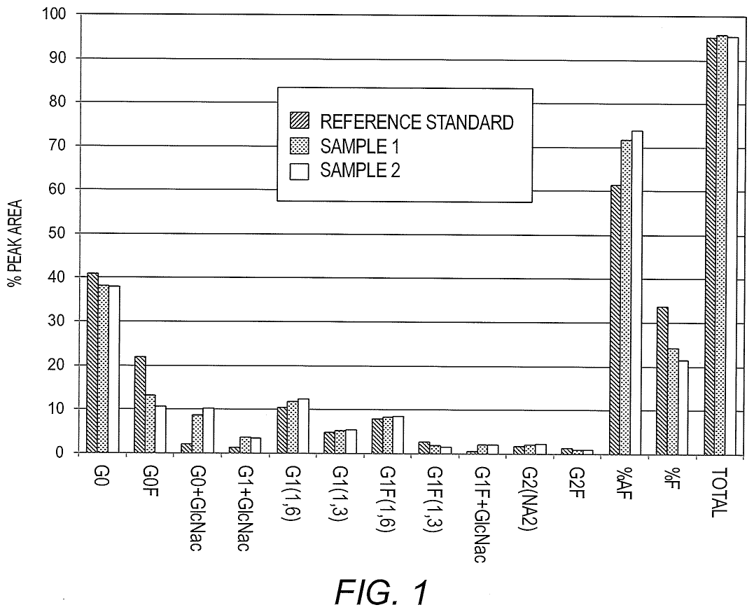

Glycoprofiling was performed to investigate oligosaccharide distribution of antibody product upon harvest from fed-batch cultures. N-glycan profiles of clarified samples were determined by conventional methods, e.g., proteolytic digestion and matrix-assisted laser desorption/ionization-mass spectrometry (MALDI-MS) or electrospray ionization-mass spectrometry ESI-MS. See, e.g., Reusch, et al. (2013) Anal. Biochem. 432:82-9; Selman, et al. (2010) Anal. Chem. 82:1073-81; Shah, et al. (2014) J. Am. Soc. Mass Spectrom. 25:999-1011; Wuhrere, et al. (2005) Anal Chem. 77:886-94; Chevreux, et al. (2011) Anal. Biochem. 415(2):212-4. Fc fragment signals with masses corresponding to N-glycan isoforms G0, G0F, G0+GlcNac, G1+GlcNac, G1(1,6), G1(1,3), G1F(1,6), G1F(1,3), G1F+GlcNac, G2(NA2), G2F were investigated, and their relative abundance rates were estimated from the intensity of the signals. The results of this analysis are presented in FIG. 1.

### Example 4: Purification and Formulation of Hu14.18K322A

Hu14.18K322A antibody purification is achieved by clarifying the cell culture broth using a depth filtration filter (e.g., Sartorius Sartoclear® PB1 Drum L filter) or by centrifugation (batch or continuous) to remove cells and cell debris. The clarified broth is then loaded onto the first column, a Protein A column (e.g., MabSelect™ PrismA). After the loading step, the Protein A column is washed with phosphate buffered saline (PBS) including 1.5 M NaCl, and 0.1 M Na Citrate, pH 6.0, which elutes host proteins and host nucleic acids. Hu14.18K322A is eluted with 0.5 M Na Citrate, pH 3.0. The Hu14.18K322A elution peak is collected and held for 30 minutes at room temperature (i.e., a low pH viral inactivation step) and is then diluted 1-fold with 35 Mm Na Acetate (NaAc), pH 4.5. The product is loaded onto a Capto™ SP ImpRes column, washed with 35 mM NaAc, pH 4.5 and 35 mM NaAc pH4.5+225 mM NaCl, and Hu14.18K322A is eluted with 35 mM NaAc, pH 4.5+600 mM NaCl. The Hu14.18K322A product pool is diluted to 7 mg/mL with 20 mM Bis Tris Propane, pH 6.8 and the buffer is exchanged into 20 mM BIS-TRIS Propane, pH 6.8 by constant volume tangential flow filtration (TFF) using a 30,000 molecular weight cut off ultrafiltration membrane. The Hu14.18K322A is filtered through a nanofilter such as that sold under the trademark Viresolve® NFP (virus removal) and then an ion exchange membrane such as that sold under the trademark a Sartobind® Q to remove residual host nucleic acids and host cell proteins. The Hu14.18K322A product pool is diafiltered into the final formulation buffer, PBS (pH 6.0) with 100 mM arginine hydrochloride with eight diafiltration volumes and concentrated to 10.5 mg/L. Polysorbate 80 was added to a final concentration of 0.03 percent w/w. The hu14.18K322A concentration was measured by UV280 nm and diluted to 10.0 mg/L. Subsequently, the diluted antibody was filtered through a 0.1 mm sterilizing grade filter sold under the trademark Sartopore® into a bioprocess bag and stored at 2-8° C.

### Example 5: Hu14.18K322A Antibody ADCC Activity

Antibody Preparation. A PROMEGA ADCC reporter bioassay is used to assess ADCC activity. A diluted stock of antibody is prepared by diluting the antibody to 1:1000 using the ADCC Assay buffer. For testing, the diluted stock is further diluted to 1 μg/mL using the ADCC Assay buffer.

Cell Preparation. Twenty hours after seeding, M21 cells are removed from the tissue culture incubator. From each well, 95 pL of media is removed and replaced with 25 pL of pre-warmed ADCC Assay buffer. To each well containing the target M21 cells is add 25 pL of diluted antibody.

Effector Cells. Effector cells are thawed in a 37° C. water bath for 2-3 minutes, followed by pipetting 630 pL of the cells into 3.6 mL of pre-warmed ADCC Assay buffer. The cells are mixed by gently pipetting 1-2 times in the assay buffer. Effector cells are transferred to sterile reagent reservoir and 25 pL are subsequently pipetted into wells containing target cells±antibody. The plates are returned the tissue culture incubator and incubated for six hours.

Preparation of Bio-Glow Luciferase Reagent. Luciferase assay buffer is thawed at room temperature and used in the reconstitution of luciferase reagent powder. After the six-hour incubation, plates are removed from the tissue culture incubator and placed at room temperature for fifteen minutes. To each of well is added 75 μL of the room temperature, reconstituted Bio-Glow reagent. Cells are incubated with the Bio-Glow reagent at room temperature for 15 minutes. The plates are incubated in the dark.

Data Acquisition. Luminescence data (RLU) is acquired at 527 nm using the Luminescent ELISA on Spectromax L SOFTMAX Pro 5.4 program. RLUs are plotted against the antibody dose (ng/mL) tested. After background subtraction, data are graphed and fit to a four-parameter logistic model equation within SOFTMAX PRO. The EC₅₀ (ng/mL) is calculated from the equation and represented by the “C” fit parameter in the equation.

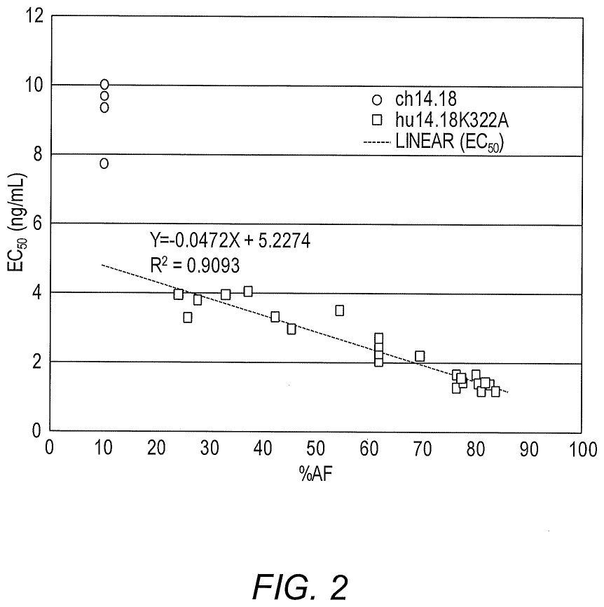

Using such an assay, ADCC data show that 96% fucosylated hu14.18K322A (expressed from NSO cells) has an ADCC activity of 146 ng/mL. By comparison, 92-94% fucosylated ch14.18 (a chimeric anti-GD2 antibody) has an ADCC activity between 8-10 ng/mL. In this same assay, an antibody with 75 to 85% afucosylation has an EC₅₀ of 1.2 to 1.5 ng/mL; 55 to 60% afucosylation has been shown to exhibit an ADCC activity of 2.1 to 2.3 ng/mL; and an antibody with 22% to 30% afucosylation has been shown to exhibit an ADCC activity of 3.5-4 ng/mL. A plot of ADCC activity versus % afucosylation is presented FIG. 2.

1: A fed-batch process for producing Hu14.18K322A monoclonal antibody in mammalian host cell culture comprising culturing mammalian host cells, which harbor a nucleic acid encoding Hu14.18K322A monoclonal antibody and are selected for producing substantially afucosylated Hu14.18K322A monoclonal antibody, in a cell culture medium comprising plant protein hydrolysates and a stable glucose concentration of 0.5 g/L to 1.5 g/L, thereby producing Hu14.18K322A monoclonal antibody in mammalian cell culture.

2: The fed-batch process of claim 1, further comprising the step of purifying the Hu14.18K322A monoclonal antibody.

3: The fed-batch process of claim 2, wherein the Hu14.18K322A monoclonal antibody is purified by contacting the cell culture medium comprising the Hu14.18K322A monoclonal antibody with a protein A resin and eluting the Hu14.18K322A monoclonal antibody.

4: The fed-batch process of claim 1, wherein the Hu14.18K322A monoclonal antibody is at least 55% afucosylated.

5: A population of substantially afucosylated Hu14.18K322A monoclonal antibodies produced by the method of claim 1.

6: The population of substantially afucosylated Hu14.18K322A monoclonal antibodies of claim 5, wherein said antibodies are at least 55% afucosylated.

7: The population of Hu14.18K322A monoclonal antibodies of claim 5, wherein said population has an antibody titer of at least about 400 mg/L.

8: The population of Hu14.18K322A monoclonal antibodies of claim 5, wherein said Hu14.18K322A monoclonal antibodies exhibit enhanced ADCC activity.

9: A pharmaceutical composition comprising the population of Hu14.18K322A monoclonal antibodies of claim 5 in admixture with a physiologically acceptable diluent, carrier, or excipient.

10: A mammalian host cell harboring a nucleic acid encoding Hu14.18K322A monoclonal antibody and selected for producing substantially afucosylated Hu14.18K322A monoclonal antibody.

11: The mammalian host cell of claim 10, wherein said antibody is at least 55% afucosylated.

12: A mammalian host cell deposited under American Type Culture Collection accession number PTA-125712 on Feb. 13, 2019.
